(19) United States
(12) Patent Application Publication
(10) Pub. No.: US 2004/0071657 A1
(43) Pub. Date: Apr. 15, 2004

(54) Chemoattractant recruitment of dendritic cells for enhancement of immunization

(76) Inventors: Charles R. Maliszewski, Seattle, WA (US); Eric A Butz, Seattle, WA (US); Laurent J Galibert, Seattle, WA (US); Luis G Borges, Oak Park, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/416,937

(22) PCT Filed: Nov. 14, 2001
(86) PCT No.: PCT/US01/46598

(51) Int. Cl. A61K 39/00; A61K 38/19; A61K 38/12

(52) U.S. Cl. 424/85.1; 424/185.1; 514/9

(57) ABSTRACT

The immune response to an antigen is enhanced through increasing the concentration of dendritic cells at a targeted site, which site may be the site of antigen contact, or may be the site of antigen presentation to T cells. Dendritic cells are attracted to the targeted site by a localization factor, which factor may be chemokine, cytokine, somatostatin receptor agonists, and the like. The methods may further be practised in conjunction with the expansion of functional dendritic cells in vivo, for example through administration of Flt3-L, GM-CSF, and the like. Also included is the use of maturation factors following antigen acquisition by the dendritic cells.

The mammalian immune system provides a springboard for much of modern medicine through its ability to raise a specific response against undesirable targets in the body. However, there are still conditions where an effective immune response has been difficult to generate. One factor that can lead to an inadequate immune response is a scarcity of antigen presenting cells at the appropriate location. T cells cannot effectively respond to antigen unless the antigen is processed and presented to them by the appropriate antigen presenting cells. The 3 major classes of antigen presenting cells are dendritic cells (DC), macrophages, and B cells, but dendritic cells are considerably more potent on a cell-for-cell basis, and are also uniquely capable of processing and presenting antigens to naive T cells. There is substantial interest, for example, in generating human-derived DC for antigen-specific tumor vaccines to be used as adjuvant immunotherapy.

A key molecule in the development of dendritic cells is Flt3 ligand (Flt3-L), which has been shown to promote DC growth. In vivo administration of Flt3-L to mice results in preferential development, mobilization or release of DC precursors from the bone marrow to the periphery and into lymphoid organs, and can increase the number of circulating DC 10-30 fold (Maraskovsky et al. (1996) J. Exp. Med. 184:1953-1962). It has also been shown to have a similar effect in the expansion of functionally competent human DC in vivo (Maraskovsky et al. (2000) Blood 96(3):878-84). The addition of GM-CSF will independently facilitate the growth of DC, and in conjunction with Flt3 will further increase the yield of functional DC (Avigan et al. (1999) Clinical Cancer Research 5(10):2735-41).

DC precursors migrate from bone marrow and circulate in the blood to specific sites in the body, where they mature. This trafficking is directed by expression of chemokine receptors and adhesion molecules. Upon exposure to antigen and activation signals, the DC are activated, and leave tissues to migrate via the afferent lymphatics to the T cell rich paracortex of the draining lymph nodes. The activated DC then secrete chemokines and cytokines involved in T cell homing and activation, and present processed antigen to T cells. This link between DC traffic pattern and functions has led to the investigation of the chemokine responsiveness of DC during their development and maturation. Chemokines are a subclass of cytokines, which have distinct structural features and biological effects. Their primary activity appears to be on the chemotaxis of leukocytes. All chemokines bind to members of a G-protein coupled serpentine receptor superfamily that span the leukocyte cell surface membrane seven times (7-TM). A review of known chemokines may be found in Rossi (2000) Annual Review of Immunology 18:217-42. For a review of the effect of chemokines on dendritic cell subsets, see Dieu-Nosjean (1999) J. Leuk. Biol. 66(2):252-62.

Chemokines, along with adhesion molecules, are crucial during inflammatory responses for a timely recruitment of specific leukocyte subpopulations to sites of tissue damage. However, chemokines and their receptors are also important in dendritic cellmaturation. For example, Fushimi (2000) J. Clin. Invest. 105(10):1383-93 explores the use of MIP-3α on the local accumulation of dendritic cells and anti-tumor immunity; and Vicari et al. (2000) J. Immunol. 165:1992 test the antitumor effects of the mouse chemokine 6Ckine/SLC. In addition to chemokines, cytokines may modulate the migration of dendritic cells (Wang et al. (1999) J. Leuk. Biol. 66(1):33-9), including interleukin (IL)-1, tumor necrosis factor alpha, and IL-10. Both the presence of the chemokines and cytokines, and the expression of the appropriate receptors may be involved. (Mantovani et al. (1998) Eur. Cytokine Network 9(3 Suppl):76-80).

Somatostatin is a tetradecapeptide that was first isolated from hypothalamic extracts and shown to be a potent inhibitor of growth hormone secretion from the anterior pituitary. Subsequent studies showed that it is widely distributed, occurring in the central nervous system and peripheral tissues such as stomach, intestine, and pancreas. Somatostatin acts at multiple sites to inhibit the release of many hormones and other secretory proteins. In addition, it functions as a neuropeptide affecting the electrical activity of neurons. Somatostatin exerts its biologic effects by binding to specific high-affinity receptors, which appear in many cases to be coupled to GTP-binding proteins.

The manipulation of dendritic cell location in the body, particularly in combination with the use of the cells in immune responsiveness, is of great interest for its potential to provide for improved methods of immunization. The present invention addresses these methods.

### SUMMARY OF THE INVENTION

Methods are provided for enhancing the immune response to an antigen by a mammalian host, through increasing the localization of dendritic cells at the targeted site. Dendritic cells are attracted to the targeted site by introduction of a localization factor. The targeted site may be the initial site of immunization where antigen is introduced to the host, or may be secondary sites, such as peripheral lymph nodes and Peyer's patches, where dendritic cell and T cell interactions take place. Factors of interest for localization include chemokines, cytokines, somatostatin receptor agonists, and the like. The methods may further be practiced in conjunction with the expansion of functional dendritic cells in vivo, for example through administration of Flt3-L, GM-CSF, and the like.

In one embodiment of the invention, the localization factor is an agonist of a somatostatin receptor. Somatostatin receptors of particular interest include one or both of the somatostatin receptors SSTR1 and SSTR3, which are found to be upregulated on activated dendritic cells. Ligands for somatostatin receptors include the native polypeptides: somatostatin and cortistatin, or may be derivatives thereof, or may be synthetic agonists, e.g. peptide and non-peptide ligands as are known in the art, or as determined by screening assays.

The methods of the invention are particularly useful in situations where the host response to the antigen is sub-optimal, for example in conditions of chronic infection, a lack of immune response to tumor antigens, and the like. In one aspect of the invention, the antigen is a tumor antigen, and is used to enhance the host immune response to tumor cells present in the body. In another aspect of the invention, the antigen is other than a tumor antigen, e.g. viral antigens, bacterial antigens, parasite antigens, etc.

### DESCRIPTION OF THE SPECIFIC EMBODIMENTS

The specific immune response for an antigen of interest is enhanced by increasing the site specific concentration of dendritic cells. A localization factor that attracts dendritic cells is introduced at the target site, where the target site may be the site of immunization, or a secondary lymphoid organ, e.g. Peyer's patches, lymph nodes, etc. The localization factor may be selected to enhance specific subsets of dendritic cells, e.g. activated dendritic cells, precursor dendritic cells, etc. Factors of interest for localization include chemokines, cytokines, somatostatin receptor agonists, and the like. The methods may further be practiced in conjunction with the expansion of functional dendritic cells in vivo, for example through administration of Flt3-L, GM-CSF, and the like.

Activated dendritic cells are surprisingly found to express somatostatin receptors, and in one embodiment of the invention, the localization factor is an agonist of a somatostatin receptor. Of particular interest are the somatostatin receptors SSTR1 and SSTR3. Ligands for somatostatin receptors include the native polypeptides somatostatin and cortistatin and derivatives thereof, or may be synthetic agonists, e.g. peptide and non-peptide ligands.

The antigen of interest may be delivered to peripheral tissues, e.g. skin, muscle, etc. or other localized sites, e.g. lymph nodes, Peyer's patches, etc., and may be given as a combined formulation, or as separate formulations. The antigen may be further provided in a booster dose, in combination with other adjuvants as known in the art, etc. The methods of the invention are particularly useful in situations where the host response to the antigen is sub-optimal, for example in conditions of chronic infection, a lack of immune response to tumor antigens, and the like.

Mammalian species that may require enhancement of T cell mediated immune responses include canines; felines; equines; bovines; ovines; etc. and primates, particularly humans. Animal models, particularly small mammals, e.g. murine, lagomorpha, etc. may be used for experimental investigations. Animal models of interest include those involved with the immune responses to infection and tumors.

### DEFINITIONS

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

Dendritic cell. As used herein, the term refers to any member of a diverse population of morphologically similar cell types found in lymphoid or non-lymphoid tissues. Dendritic cells are a class of “professional” antigen presenting cells, and have a high capacity for sensitizing MHC-restricted T cells. Dendritic cells may be recognized by function, or by phenotype, particularly by cell surface phenotype. These cells are characterized by their distinctive morphology, high levels of surface MHC-class II expression and ability to present antigen to T cells, particularly to naive T cells (Steinman et al. (1991) Ann. Rev. Immunol. 9:271; incorporated herein by reference for its description of such cells). The dendritic cells affected by the methods of the invention may be selected to be immature or mature dendritic cells.

The cell surface of dendritic cells is unusual, with characteristic veil-like projections, and is characterized by expression of the cell surface markers CD1a⁺, CD4⁺, CD86⁺, or HLA-DR⁺. Mature dendritic cells are typically CD11c⁺, while precursors of dendritic cells include those having the phenotype CD11c⁻, IL-3Rα^{low}; and those that are CD11c⁻ IL-3Rα^{high}. Treatment with GM-CSF in vivo preferentially expands CD11b^{high}, CD11c^{high} DC, while Flt-3 ligand has been shown to expand CD11c⁺ IL-3Rα^{low} DC, and CD11c⁻ IL-3Rα^{high} DC precursors.

Functionally, dendritic cells maybe identified by any convenient assay for determination of antigen presentation. Such assays may include testing the ability to stimulate antigen-primed or naive T cells by presentation of a test antigen, following by determination of T cell proliferation, release of IL-2, and the like.

Dendritic cell localization factor. As used here, localization factors are compounds that enhance the site specific concentration of dendritic cells in vivo. Localization factors of interest include chemokines, e.g. MIP-3α; MIP-3β, MIP-5, MDC, SDF-1, MCP-3, MCP-4, and Rantes. Immature DC respond to many CC- and CXC-chemokines (MIP-1alpha, MIP-1beta, MIP-5, MCP-3, MCP-4, RANTES, TECK, and SDF-1) and in particular to MIP-3alpha/LARC, which acts through the receptors CCR1; CXCR4; and CCR6, a receptor mainly expressed in DC and lymphocytes. In contrast, mature DC have lost their responsiveness to most of these chemokines through receptor down-regulation or desensitization, but have acquired responsiveness to MIP-3beta/ELC and 6Ckine/SLC as a consequence of CCR7 expression. Cytokines, e.g. interleukin (IL)-1, tumor necrosis factor alpha, and IL-10, are also capable of acting as a localization factor. Derivatives, analogs and mimetics of any of these compounds may also be used. Localization factors of particular interest include agonists and ligands for these factors, as well as for somatostatin receptors, as described in detail below.

The localization factor may be delivered as a bolus, or may provide for a localized concentration by use of a sustained release formulation. For example, it may be desirable to increase the number of dendritic cells at a target site prior to antigen administration. Alternatively, the antigen and localization factor may be co-administered. Preliminary doses can be determined according to animal tests, and the scaling of dosages for human administration can be performed according to art-accepted practices.

A variety of sustained release formulations are known and used in the art. For example, biodegradable or bioerodible implants may be used. The implants may be particles, sheets, patches, plaques, fibers, microcapsules and the like and may be of any size or shape compatible with the selected site of insertion. Characteristics of the polymers will include biodegradability at the site of implantation, compatibility with the agent of interest, ease of encapsulation, the half-life in the physiological environment, water solubility, and the like.

Another approach involves the use of an implantable drug delivery device. Examples of such implantable drug delivery devices include implantable diffusion systems (see, e.g., subdermal implants (such as NORPLANT™) and other such systems, see, e.g., U.S. Pat. Nos. 5,756,115; 5,429,634; 5,843,069). These implants generally operate by simple diffusion, e.g., the active agent diffuses through a polymeric material at a rate that is controlled by the characteristics of the active agent formulation and the polymeric material. Alternatively, the implant may be based upon an osmotically-driven device to accomplish controlled drug delivery (see, e.g., U.S. Pat. Nos. 3,987,790, 4,865,845; 5,057,318; 5,059,423; 5,112,614; 5,137,727; 5,234,692; 5,234,693; and 5,728,396). These osmotic pumps generally operate by imbibing fluid from the outside environment and releasing corresponding amounts of the therapeutic agent.

Chemokine or Somatostatin receptor agonists. As defined herein, refer to compounds, which may be polypeptide compounds or non-polypeptides, that bind to one or more of the chemokine receptors, as described above, or to the family of mammalian somatostatin cell surface receptors SSTR1, SSTR2, SSTR3, SSTR4 and SSTR5, or homologs or orthologs thereof, and activate the receptor. Known somatostatin receptors include the human proteins as described by Yamada et al. (1992) P.N.A.S. 89:251-255; Rohrer et al. (1993) P.N.A.S. 90: 4196-4200; and Yamada et al. (1993) Biochem Biophys Res Commun. 195(2):844-52. The sequences of these proteins are publicly known and available, e.g. in Genbank. The accession numbers for the amino acid sequences are as follows: SSTR1 has Genbank accession number A41795; SSTR2 has Genbank accession number B41795; SSTR3 has Genbank accession number AAA60592; SSTR4 has Genbank accession number AAA60565; SSTR5 has Genbank accession number JN0763. Exemplary somatostatin receptors based on the above proteins include are the SSTR1 and SSTR3 receptors, as described above, and homologs and orthologs thereof.

Several naturally occurring polypeptides are ligands for somatostatin receptors. These include the somatostatin peptides SST-14 and SST-28; and cortistatin peptides CST-17 and CST-29 (see Bruns et al. (1996) Metabolism 45(suppl 1):17-20; and Patel (1997) J. Endocrin. Invest 20:348-367). The native human somatostatin SST-28 peptide has the sequence SANSNPAMAPRERKAGCKNFFWKTFTSC (SEQ ID NO: 1), and the SST-14 peptide has the sequence AGCKNFFWKTFTSC (SEQ ID NO: 2). Human CST-29 has the sequence QEGAPPQQSARRDRMPCRNFFWKTFSSCK (SEQ ID NO: 3) and CST-17 has the sequence DRMPCRNFFWKTFSSCK (SEQ ID NO: 4).

Other known peptide agonists include the short synthetic peptides octreofide, lanreotide, vapreotide, seglitide, BIM23268, NC8-12, BIM23197, CH275, etc. (Bruns et al., supra.). The cortistatin peptides have been found to have a high affinity for SSTR3, as does the synthetic peptide NC8-12. Somatostatin, and the synthetic peptide CH275 have a higher binding affinity for the SSTR1 receptor.

The sequence of the polypeptides may be altered in various ways known in the art to generate targeted changes in sequence. A variant polypeptide will usually be substantially similar to the sequences provided herein, i.e. will differ by at least one amino acid, and may differ by at least two but usually not more than about four amino acids. The sequence changes may be substitutions, insertions or deletions. Scanning mutations that systematically introduce alanine, or other residues, may be used to determine key amino acids. Amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids.

The peptides may be prepared by in vitro synthesis, using conventional methods as known in the art. Various commercial synthetic apparatuses are available, for example, automated synthesizers by Applied Biosystems Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. For example, a lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usuallyat least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Other molecules that interact with the somatostatin receptors may be used in the subject methods. For example, known non-peptide agonists include L-797,591; L-779,976; L-796,778; L-803,087; L-817,818; etc. (see Rohrer et al. (1998) Science 282:737-740). For example, it has been found that L-797,591 has a high degree of specificity for SSTR1, while L-796,778 has a higher affinity for SSTR3.

In addition to compounds known in the art, candidate agonists may be screened for their ability to bind to, and activate, a somatostatin receptor. Assays to determine affinity and specificity of binding are known in the art, including competitive and non-competitive assays. Assays of interest include ELISA, RIA, immunoblots, flow cytometry, etc. Binding assays may use purified or semi-purified SSTR1 or SSTR3 protein, or alternatively may use cells that express a somatostatin receptor, e.g. cells transfected with an expression construct for SSTR1 or SSTR3, etc. As an example of a binding assay, purified SSTR1 or SSTR3 receptor protein is bound to an insoluble support, e.g. microtiter plate, magnetic beads, etc. The candidate agonist and soluble, labeled somatostatin are added to the cells, and the unbound components are then washed off. The ability of the agonist to compete for SSTR1 or SSTR3 receptor binding is determined by quantitation of bound, labeled somatostatin. A functional assay that detects chemotaxis of dendritic cells may be used for confirmation.

Suitable agonists, in addition to those described above and variants thereof, include peptides, small organic molecules, peptidomimetics, antibodies, or the like. Antibodies may be polyclonal or monoclonal; intact or truncated, e.g. F(ab′)₂, Fab, Fv; xenogeneic; allogeneic; syngeneic; or modified forms thereof, e.g. humanized, chimeric, etc.

In many cases, the agonist will be a polypeptide, e.g. somatostatin, an antibody or fragment thereof, etc., but other molecules that provide relatively high specificity and affinity may also be employed. Combinatorial libraries provide compounds other than oligopeptides that have the necessary binding characteristics.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries- and compounds are readily modified through conventional chemical, physical and biochemical means. Known pharmacological agents may be subjected to directed or random chemical modifications, such as acylation, alkylation, esterification, amidification to produce structural analogs.

Suitable antibodies for use as agonists are obtained by immunizing a host animal with peptides comprising all or a portion of a somatostatin receptor, etc. Suitable host animals include mouse, rat, sheep, goat, hamster, rabbit, etc. The origin of the protein immunogen may be mouse, human, rat, monkey, etc. The host animal will generally be a different species than the immunogen, e.g. mouse SSTR1 or SSTR3 used to immunize hamsters, human SSTR1 or SSTR3 to immunize mice, etc. Methods to generate monoclonal antibodies are well known in the art and need not be further elaborated.

Expansion of dendritic cells. In combination with the local recruitment of dendritic cells, the overall number of functionally mature dendritic cells in the host may be expanded through the prior administration of a suitable growth factor, which growth factor may be one or more of Flt3-L; GM-CSF; G-CSF; GM-CSF+IL4; GM-CSF +IL-3; etc.

For example, flt3-L has been found to stimulate the generation of large numbers of functionally mature dendritic cells, both in vivo and in vitro (U.S. Ser. No.08/539,142, filed Oct. 4, 1995). Flt3-L refers to a genus of polypeptides that are described in EP 0627487 A2 and in WO 94/28391, both incorporated herein by reference. A human flt3-L cDNA was deposited with the American Type Culture Collection, Rockville, Md., USA (ATCC) on Aug. 6, 1993 and assigned accession number ATCC 69382. Other useful cytokines include granulocyte-macrophage colony stimulating factor (GM-CSF; described in U.S. Pat. Nos. 5,108,910, and 5,229,496 each of which is incorporated herein by reference). Commercially available GM-CSF (sargramostim, Leukine®) is obtainable from Immunex Corp., Seattle, Wash.) Moreover, GM-CSF/IL-3 fusion proteins (i.e., a C-terminal to N-terminal fusion of GM-CSF and IL-3) may be used. Such fusion proteins are well known in the art and are described in U.S. Pat. Nos. 5,199,942; 5,108,910 and 5,073,627, each of which is incorporated herein by reference.

Various routes and regimens for delivery may be used, as known and practiced in the art. For example, where the agent is Flt3-L, the Flt3-L may be administered daily, where the dose is from about 1 to 100 mg/kg body weight, more usually from about 10 to about 50 mg/kg body weight. Administration may be at a localized site, e.g. sub-cutaneous, or systemic, e.g. intraperitoneal, intravenous, etc.

Antigens. A wide variety of antigens benefit from the methods of the present invention. Antigens of interest include polypeptides and other immunogenic biomolecules, which may be isolated or derived from natural sources, produced by recombinant methods, etc., as known in the art. Alternatively complex antigens may be used, such as whole protein cell lysates, RNA, plasmids, whole cells, heat shock proteins, virus which may be inactivated, bacterial cells or fractions derived therefrom, and the like.

For example, dendritic cell recruitment methods may be used in conjunction with vaccines such as, but not limited to, those for treating chronic bacterial infections, e.g. tuberculosis, etc.; chronic viral infections such as those associated with herpesvirus, lentivirus and retrovirus, etc. Antigens of interest may also include allergens, e.g. for the conversion of a Th2 to a Th1 type response; or antigens for the conversion of autoimmune Th1 response to Th2 response. The antigens which may be incorporated into the present formulations include viral, prokaryotic and eukaryotic antigens, including but not limited to antigens derived from viruses, bacteria, fungi, protozoans, parasites and tumor cells.

Potential tumor antigens for immunotherapy include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and over-expressed self antigens, e.g. her-2/neu; carcinoembryonic antigen, muc-1, and the like.

Tumor cell derived protein extracts, exosomes or RNA may be used as a source of antigen, in order to provide multiple antigens and to increase the probability of inducing immunity to more than one tumor associated antigen. Although the target antigens are initially undefined, the relevant specific antigen can be later identified.

A number of antigens expressed on normal tissues as well as tumors are useful as immunotherapy targets, and have been shown to stimulate T cell responses when the antigens are presented by dendritic cells.

Antigenic formulations will typically contain from about 0.1 μg to 1000 μg, more preferably 1 μg to 100 μg, of the selected antigen. The antigen composition may additionally contain biological buffers, excipients, preservatives, and the like.

The antigen is administered to the host in the manner conventional for the particular immunogen, generally as a single unit dose of an antigen in buffered saline, combined with the adjuvant formulation, where booster doses, typically one to several weeks later, may additionally be delivered enterally or parenterally, e.g., subcutaneously, intramuscularly, intradermally, intravenously, intraarterially, intraperitoneally, intranasally, orally, etc. Subcutaneous or intramuscular injection is, however, preferred.

Methods of Use

To enhance the efficacy of immunization in a mammalian host, the concentration of dendritic cells is increased at a targeted location, through the site specific use of a dendritic cell localization factor. The localization factor may be included with the dose of immunogen, may be administered in a separate formulation at the same site, or may be administered at a site of antigen presentation to T cells, such as lymph nodes, Peyer's patches, and the like.

Where the antigen and localization agent are administered at the same site, the localization may be performed before, during or after the immunization. Either or both of the immunogen and the localization factor may be formulated for site specific delivery, e.g. implants and other sustained release formulations. Where the antigen is administered at, e.g., a peripheral site and the dendritic cells are localized to, e.g., a lymph node, then the localization step will generally follow the immunization step, in order to permit the dendritic cells to acquire peripheral antigen prior to migration to the lymph node.

To further increase the number of available dendritic cells, prior to immunization the total number of competent dendritic cells may be expanded through the systemic or local administration of an expansion factor such as Flt3-L, GM-CSF and the like, generally prior to the localization and immunization step.

By increasing the local concentration of functional dendritic cells at the site of action, the response to immunogens that are poorly recognized by the immune system is enhanced. These methods are of particular value for antigens such as viral and bacterial antigens associated with chronic infection, tumor antigens, etc., and for the response of immunocompromised animals, where an effective immune response is difficult to obtain.

Experimental

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### EXAMPLE 1

Expression of Somatostatin Receptors by Activated Dendritic Cells

Materials and Methods

PCR. Dendritic Cells (DC) were prepared from the spleens and/or lymph nodes of mice treated with Flt3-L (10 days at 10 μg/day) or GM-CSF (pegylated, 5 days at 2 μg/day, sc), both of which can expand DC populations in mice and humans (Maraskovsky (2000); Avignan (1999), supra.) In some cases mice were also treated with LPS, TNF, poly I:C, or CD40L-LZ to activate the DC in vivo. After cytokine treatment, spleens and/or lymph nodes were collected and dendritic cells (DC) were prepared by density gradient centrifugation and flow cytometry, following which RNA was prepared from these cells using Qiagen Rneasy® colums according to the manufacturer's directions. This RNA was then treated with Ambion® Rnase-free Dnase to eliminate any contaminating genomic DNA in the samples. Control experiments were performed to ensure that this treatment did not damage the RNA samples. To assay for the presence of chemokine receptor mRNA, cDNAs were prepared using an Applied BioSystems first strand synthesis kit, and random hexanuleotide primers followed by real time PCR amplification of transcripts for the following chemokine receptors: CCR1, CCR2, CCR3, CCR4, CCR5, CCR6, CCR7, CCR8, CCR9, CXCR1, CXCR4, CXCR5; each in conjunction with a multiplexed housekeeping gene (β actin, GAPDH, or PBGD) was performed. Primers for these reactions were purchased from Applied Biosystems.

Alternatively, DNAse-treated RNA samples are used to probe Affymetrix® GeneChip® arrays of selected mouse or human genes, or in-house generated arrays of DC-derived genes, both of which include chemokine and chemokine receptor targets.

In vitro assay of chemotaxis. Chemotaxis is measured in vitro by the ability of purified chemokines or somatostatin to promote migration of Calcein-AM (Molecular Probes; Eugene, Oreg.) tagged murine and human DC through FALCON HTS 3™ Fluoroblok membrane inserts (Becton Dickinson Labware; Franklin Lakes, N.J.) in multiwell plates. The number of cells migrating through the membranes (chemotactic toward the stimulus) are quantitated by serial measurements on a Cytofluor 2 (PerSeptive Biosystems; Framingham Mass.).

In vivo assay of chemotaxis. The abdomens of anesthetized mice (some previously treated with Flt3-L or GM-CSF (pGM)) are shaved and the test agent (TA)-loaded CollaPlug® (Calcitek, Plainsboro, N.J., USA) is inserted subcutaneously. At various times thereafter the mice are sacrificed and the collagen plug recovered for histological or flow cytometric analysis (flow cytometric analysis is done following collagenase disruption of the plug to release cells that had migrated into it). In this context the test agent is a purified chemokine, or somatostatin or a mix of such agents, with or without antigen, and with or without one or more DC activating agent(s) such as CD40L, IL-12, IFNγ, etc.

DC Preparation from FltI3L or GM-CSF treated mice using density gradient (Nycodenz). Spleens and lymph nodes (inguinal, axillary, brachial and mesenteric nodes) were removed from treated mice (Flt3-L 10 μg/mouse/day, 9 days; peg-GM-CSF 5 μg/mouse/day, 6 days).

100 U/ml collagenase was injected (2 ml/Spleen, 1 ml/Sp for untreated mice) of with a 5 or 10 ml syringe in a 100 mm dish. Large LN were similarly injected, smaller LN were shredded open with forceps and incubated in collagenase solution. Any cells that washed out of the tissue during the injection/shredding steps were collected into a 50 ml tube with HBSS/EDTA and put on ice. 2 ml/spleen (1 ml/spleen for untreated mice) of 400 U/ml collagenase was added to the dish with the spleens, covered, and incubated at 37° C./5% CO2/100% humidity for 30 min.

The collagenase solution was removed, and 1 ml/spleen of HBSS/EDTA was added, then mashed using 3 ml syringe plunger and stainless steel wire mesh screens. The suspension was pipetted vigorously up and down in a 10 ml pipet and used to filter out any large fragments that remained. The released cells were pooled with the cells set aside during the collagenase injections and centrifuged 10 min. at 1500 rpm/4° C.

The supernatant was removed and the cells resuspended in HBSS/EDTA. The suspension was pipetted vigorously up and down in a 10 ml pipet to break up any cell clumps, then spun down.

The cells were resuspended in Nycodenz solution, 1-2 spleens/tube, (5-6 spleens/tube for untreated Sp/LNC), and overlayed with 3ml/tube HBSS/EDTA, then centrifuged for 15 min at 1700×G/4° C. without a brake.

The cells at the interface were collected and washed in complete medium.

FACS Sorting of DC Populations

The concentration of depleted cells was adjusted to 10⁸/ml, and the following sets prepared:

| | | |
|---|---|---|
| 10 ⁵-10⁶ cells | no antibody | (4 ml FACS tube) |
| 10 ⁵-10⁶ cells | αCD11b-FITC + 2.4G2 | (4 ml FACS tube) |
| 10 ⁵-10⁶ cells | αCD11c-PE + 2.4G2 | (4 ml FACS tube) |
| remaining cells | αCD11b-FITC αCD11c-PE + 2.4G2 | (15 ml tube) |

The minimum volume in each tube was 50 μl. The 2.4G2 was added at a dilution of 1:50 and the PE- and FITC-conjugated antibodies were added at a previously determined optimal dilution (for Pharmingen antibodies, typically ˜1:100). The cells were incubated on ice for 25 minutes, then washed twice with ice cold PBS/5 (2 ml washes for FACS tubes, 13 ml washes for 15 ml tubes). The concentration was adjusted for the reserved sorter (Elite: 2·10⁷/ml) and cells were transfered to 4 ml polystyrene FACS tubes. The unstained and one color controls were in 100 μl each.

All publications and patent applications cited in this specification are herein incorporated by reference as if each individual publication or patent application were specifically and individually indicated to be incorporated by reference.

Although the foregoing invention has been described in some detail byway of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of increasing the immune response in a mammalian host to a immunogen, the method comprising:
administering a dendritic cell localization agent at a target site in said host, in a dose effective to substantially increase the number of dendritic cells present at said target site;
immunizing said host with said immunogen, wherein said immunogen is introduced into said host at said target site.

2. The use of a dendritic cell localization agent in a dose effective to substantially increase the number of dendritic cells at a target site, for the manufacture of a medicament for increasing the immune response in a mammalian host to an immunogen.

3. The method according to either of claims 1 or 2, wherein said target site is cutaneous.

4. The method according to either of claims 1 or 2, wherein said target site is intramuscular.

5. The method according to either of claims 1 or 2, wherein said target site is intratumor.

6. The method according to either of claims 1 or 2, wherein said target site is a lymph node.

7. The method according to either of claims 1 or 2, wherein said target site is one of Peyer's patches, spleen or thymus.

8. The method according to either of claims 1 or 2, wherein said dendritic cell localization agent is a chemokine.

9. The method according to claim 8, wherein said chemokine is selected from the group consisting of MIP-3α; MIP-3β, MIP-5, MDC, SDF-1, MCP-3, MCP-4, TECK, and Rantes.

10. The method according to either of claims 1 or 2, wherein said dendritic cell localization agent is an agonist of a somatostatin receptors.

11. The method according to claim 10, wherein said somatostatin receptor is at least one of SSTR1 and SSTR3.

12. The method according to claim 11, wherein said agonist is somatostatin.

13. The method of claim 12, wherein said somatostatin agonist is cortistatin or a cleavage product derived therefrom.

14. The method of claim 12, wherein said somatostatin agonist is a peptide.

15. The method of claim 10, wherein said somatostatin agonist is a non-peptide agonist.

16. The method of either of claims 1 or 2, wherein said immunogen and said dendritic cell localization agent are co-formulated.

17. The method of either of claims 1 or 2, wherein said immunogen and said dendritic cell localization agent are separately formulated.

18. The method of claim 17, wherein said dendritic cell localization agent is administered prior to said immunogen.

19. The method of either of claims 1 or 2, wherein said immunogen is a tumor antigen.

20. The method of either of claims 1 or 2, wherein said immunogen is a bacterial antigen.

21. The method of either of claims 1 or 2, wherein said immunogen is a viral antigen.

22. The method of either of claims 1 or 2, wherein said immunogen is a polypeptide.

23. The method of either of claims 1 or 2, wherein said immunogen is a nucleic acid encoding a polypeptide.

24. The method of either of claims 1 or 2, wherein said mammalian host is a human.

25. The method of claim 1, further comprising the step of expanding dendritic cells in said mammalian host by administration of at least one of Flt3-L and GM-CSF or other agents designed to expand dendritic cells.

26. A composition comprising a dendritic cell localization agent in a dose effective to substantially increase the number of dendritic cells present at a target site, for use in the immunization of a host with an immunogen.

27. The composition of claim 26, wherein said target site is cutaneous.

28. The composition of claim 26, wherein said target site is intramuscular.

29. The composition of claim 26, wherein said target site is intratumor.

30. The composition of claim 26, wherein said target site is a lymph node.

31. The composition of claim 26, wherein said target site is one of Peyer's patches, spleen or thymus.

32. The composition of claim 26, wherein said dendritic cell localization agent is a chemokine.

33. The composition of claim 32, wherein said chemokine is selected from the group consisting of MIP-3α; MIP-3β, MIP-5, MDC, SDF-1, MCP-3, MCP-4, TECK, and Rantes.

34. The composition of claim 26, wherein said dendritic cell localization agent is an agonist of a somatostatin receptors.

35. The composition of claim 34, wherein said somatostatin receptor is at least one of SSTR1 and SSTR3.

36. The composition of claim 34, wherein said agonist is somatostatin.

37. The composition of claim 34, wherein said somatostatin agonist is cortistatin or a cleavage product derived therefrom.

38. The composition of claim 34, wherein said somatostatin agonist is a peptide.

39. The composition of claim 34, wherein said somatostatin agonist is a non-peptide agonist.

40. The composition of claim 26, wherein said immunogen and said dendritic cell localization agent are co-formulated.

41. The composition of claim 26, wherein said immunogen and said dendritic cell localization agent are separately formulated.

42. The composition of claim 41, wherein said dendritic cell localization agent is administered prior to said immunogen.

43. The composition of claim 26, wherein said immunogen is a tumor antigen.

44. The composition of claim 26, wherein said immunogen is a bacterial antigen.

45. The composition of claim 26, wherein said immunogen is a viral antigen.

46. The composition of claim 26, wherein said immunogen is a polypeptide.

47. The composition of claim 26, wherein said immunogen is a nucleic acid encoding a polypeptide.

48. The composition of claim 26, wherein said mammalian host is a human.

49. The composition of claim 26, wherein said dendritic cells are expanded by administration of at least one of Flt3-L and GM-CSF or other agents designed to expand dendritic cells.


SEQUENCE LISTING (4 sequences)

SEQ ID NO: 1 - protein, 28 aa, Homo sapiens
SANSNPAMAPRERKAGCKNFFWKTFTSC

SEQ ID NO: 2 - protein, 14 aa, Homo sapiens
AGCKNFFWKTFTSC

SEQ ID NO: 3 - protein, 29 aa, Homo sapiens
QEGAPPQQSARRDRMPCRNFFWKTFSSCK

SEQ ID NO: 4 - protein, 17 aa, Homo sapiens
DRMPCRNFFWKTFSSCK